(19) United States
(12) Patent Application Publication
(10) Pub. No.: US 2026/0358281 A1
(43) Pub. Date: Nov. 12, 2026

(54) A System and Method for Estimating Total Dissolved Solids in Boiler Water

(71) Applicants: Spirax-Sarco Limited, Cheltenham (GB); The University of Nottingham, Nottingham (GB)

(72) Inventors: Michael Agolom, Cheltenham (GB); Chris Dodds, Nottingham (GB); Juliano Katrib, Nottingham (GB)

(21) Appl. No.: 18/997,507

(22) PCT Filed: Jul. 6, 2023
(86) PCT No.: PCT/EP2023/068800

§ 371 Date: Jan. 21, 2025

(30) Foreign Application Priority Data
Jul. 21, 2022 (GB) 2210666.0

(51) Int. Cl. G01N 27/06; G01N 22/00; G01N 33/18

(52) U.S. Cl. CPC G01N 27/06; G01N 22/00; G01N 33/1853

(57) ABSTRACT

A system for estimating total dissolved solids (TDS) concentration in boiler water is described which comprises: a sensor having a transmitter configured to transmit an RF or microwave signal into the boiler water and a receiver configured to receive a reflected signal from a reflection of the RF or microwave signal by the boiler water; and an analysis module configured to determine a conductivity value for the boiler water based on the reflected signal and to determine a TDS concentration for the boiler water based on the conductivity. A corresponding method is also described.

The disclosure relates to a system and method for estimating total dissolved solids in boiler water.

### BACKGROUND

As a boiler generates steam, any impurities which are in the boiler feedwater and which do not boil off with the steam will concentrate in the boiler water.

As the total dissolved solids (TDS) become more and more concentrated, the steam bubbles tend to become more stable, failing to burst as they reach the water surface of the boiler. There comes a point (depending on boiler pressure, size, and steam load) where a substantial part of the steam space in the boiler becomes filled with bubbles and foam is carried over into the steam main.

This is undesirable not only because the steam is excessively wet as it leaves the boiler, but it contains boiler water with a high level of dissolved and perhaps suspended solids. These solids can contaminate control valves, heat exchangers and steam traps.

TDS come from dissolved organic and inorganic substances in the boiler feedwater. In particular, common salt constituents in the boiler feedwater include calcium, sodium, chloride, potassium, phosphates and nitrates.

In order to avoid performance and safety issues in the boiler, there is a need to measure, monitor and control the TDS concentration in the boiler water.

According to an aspect, there is provided a system for estimating total dissolved solids (TDS) concentration in boiler water, the system comprising: a sensor having a transmitter configured to transmit an RF or microwave signal into the boiler water and a receiver configured to receive a reflected signal from a reflection of the RF or microwave signal by the boiler water; and an analysis module configured to determine a conductivity value for the boiler water based on the reflected signal and to determine a TDS concentration for the boiler water based on the conductivity.

The analysis module may be further configured to determine a reflection coefficient based on the reflected signal and to determine the conductivity value based on the reflection coefficient.

The system may further comprise a control module which is configured to determine if the TDS concentration exceeds a threshold value and to take action if the TDS concentration does exceed the threshold value.

The control module may output an alert to an operator if the TDS concentration exceeds the threshold value.

The control module may open a blowdown control valve if the TDS concentration exceeds the threshold value in order to reduce the TDS concentration.

The sensor may be an open-ended coaxial probe. The probe may be a high pressure-high temperature probe.

The analysis module may comprise a vector network analyzer.

According to another aspect, there is provided a steam boiler comprising a system as described above.

According to another aspect, there is provided a method of estimating total dissolved solids (TDS) concentration in boiler water, the method comprising: transmitting an RF or microwave signal into the boiler water; receiving a reflected signal from a reflection of the RF or microwave signal by the boiler water; determining a conductivity value for the boiler water based on the reflected signal; and determining a TDS concentration for the boiler water based on the conductivity.

The method may further comprise: determining a reflection coefficient based on the reflected signal; wherein the conductivity value is determined based on the reflection coefficient.

The RF or microwave signal may be transmitted and received using a sensor probe.

The sensor probe may be an open-ended coaxial probe. Alternatively, the RF or microwave signal may be transmitted and received using a coaxial transmission line or resonant cavity measurement system which may be connected to a blowdown line.

The method may further comprise: determining if the TDS concentration exceeds a threshold value; and taking action if the TDS concentration does exceed the threshold value.

The action may include providing an alert to an operator.

The action may include opening a blowdown control valve in order to reduce the TDS concentration.

### BRIEF DESCRIPTION OF DRAWINGS

For a better understanding of the invention, and to show more clearly how it may be carried into effect, reference will now be made, by way of example, to the accompanying drawings, in which:

FIG. 1 is a schematic of a steam boiler plant with a system according to an embodiment of the invention;

FIG. 2(a) is a front view of a probe of the system and FIG. 2(b) is a cross-sectional view of the probe; and

FIG. 3 is a flowchart of a method according to an embodiment of the invention.

### DETAILED DESCRIPTION

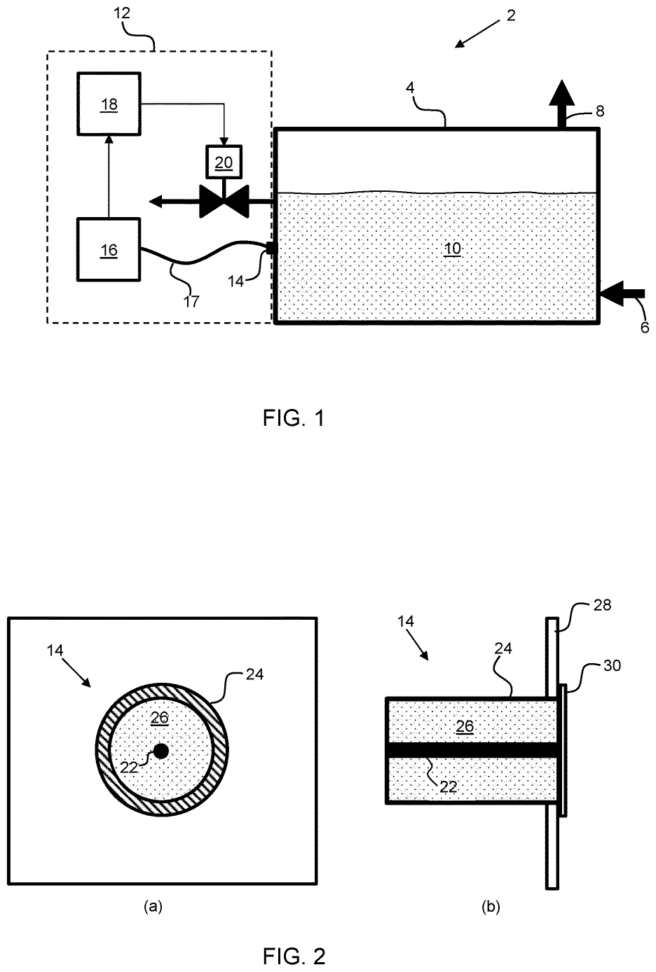

FIG. 1 shows a schematic of a steam boiler plant 2. The steam boiler plant 2 comprises a steam boiler 4. The steam boiler 4 is connected to a feedwater line 6 and a steam main 8. Water 10 is pumped to the boiler 4 from a feedtank by a feedpump (not shown). The water 10 is then heated within the boiler 4 to form steam which is released via the steam main 8 and transported to the point of use.

The steam boiler 4 is provided with a TDS monitoring system 12 according to an embodiment of the invention. The system 12 comprises a sensor probe 14 which is connected to an analysis module 16. The output from the analysis module 16 is provided to a control module 18 which may be used to control a blowdown valve 20 or take other action.

As shown in FIG. 2(a) and 2(b), the sensor probe 14 is an open-ended coaxial probe which comprises an inner conductor 22 and an outer conductor 24 which are arranged concentrically and spaced from one another by a dielectric material 26. The dielectric material 26 is disposed between the inner conductor 22 and the outer conductor 24 such that the inner conductor 22 and the outer conductor 24 are electrically isolated from one another.

As shown in FIG. 2(b), the sensor probe 14 is inserted through an opening formed in a wall 28 of the steam boiler 4 such that it is submerged in the boiler water 10. The sensor probe 14 may be a high pressure-high temperature probe which is able to withstand the conditions with the steam boiler 4. The outer conductor 24 may form a flange 28 which lies against an inner surface of the wall 28.

As described previously, the sensor probe 14 is connected to an analysis module 16 via a probe connection cable 17. In this embodiment, the analysis module 16 comprises a vector network analyser (VNA). The VNA comprises a signal source which generates a Radio Frequency (RF) or microwave signal which is transmitted to the sensor probe 14.

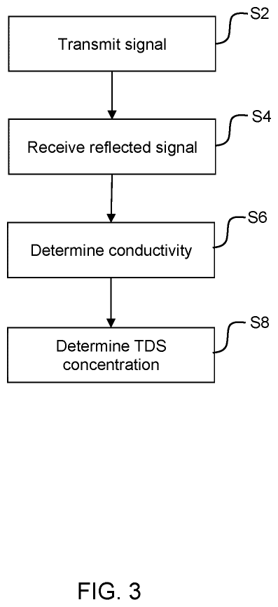

The RF or microwave signal creates an electromagnetic field between the inner conductor 22 and the outer conductor 24 which is transmitted into and reflected by the boiler water 10, as depicted by step S2 of FIG. 3. The VNA further comprises a receiver which, at step S4, receives the reflected signal from the sensor probe 14 after reflection by the boiler water 10.

At step S6, the analysis module 16 determines a conductivity value for the boiler water 10. This is determined by calculating a reflection coefficient based on the ratio of the amplitude of the reflected signal to that of the transmitted signal (i.e., incident signal). It has been found that the amplitude of the reflected signal is dependent on the conductivity of the boiler water 10. Accordingly, the analysis module 16 is then able to determine the conductivity of the boiler water 10 from the reflection coefficient. At step S10, the analysis module 16 then determines the TDS concentration from the conductivity.

Correlations between the reflection coefficient and the conductivity and between the conductivity and the TDS concentration may be determined empirically. The method may be repeated with signals having different frequencies in order to improve accuracy.

As described previously, the output from the analysis module 16 may be provided to the control module 18 which is able to take action in response to the current TDS concentration. In particular, the control module 18 may open the blowdown valve 20 in order to release water 10 from the boiler 4 which is then replaced by new feedwater in order to reduce the TDS concentration of the boiler water in the boiler 4. This action may be taken when the current TDS concentration exceeds a threshold value.

In other embodiments, the control module 18 may provide an alert, such as an audible or visual alert, which serves as a prompt for a human operator to take action in order to reduce the TDS concentration. For example, the operator may manually open the blowdown valve 20. In other embodiments, the output from the analysis module 16 may simply be displayed to an operator in order to allow them to monitor and control the TDS concentration. It will be appreciated that the analysis module 16 and control module 18 may be embodied by a single unit (e.g., a processor) and distinguished only by their functionality.

In other examples, the RF or microwave signal may be transmitted and received using a coaxial transmission line or resonant cavity measurement system which may be connected to a blowdown line.

The invention is not limited to the embodiments described herein and may be modified or adapted without departing from the scope of the present invention.

1. A system for estimating total dissolved solids (TDS) concentration in boiler water, the system comprising:
a sensor having a transmitter configured to transmit an RF or microwave signal into the boiler water and a receiver configured to receive a reflected signal from a reflection of the RF or microwave signal by the boiler water; and
an analysis module configured to determine a conductivity value for the boiler water based on the reflected signal and to determine a TDS concentration for the boiler water based on the conductivity.

2. A system as claimed in claim 1, wherein the analysis module is further configured to determine a reflection coefficient based on the reflected signal and to determine the conductivity value based on the reflection coefficient.

3. A system as claimed in claim 1, further comprising a control module which is configured to determine if the TDS concentration exceeds a threshold value and to take action if the TDS concentration does exceed the threshold value.

4. A system as claimed in claim 3, wherein the control module outputs an alert to an operator if the TDS concentration exceeds the threshold value.

5. A system as claimed in claim 3, wherein the control module opens a blowdown control valve if the TDS concentration exceeds the threshold value in order to reduce the TDS concentration.

6. A system as claimed in claim 1, wherein the sensor is an open-ended coaxial probe.

7. A system as claimed in claim 1, wherein the analysis module comprises a vector network analyzer.

8. A steam boiler system comprising:
a boiler configured to contain boiler water;
a monitoring system configured for estimating total dissolved solids (TDS) concentration in the boiler water, the monitoring system comprising:
a sensor having a transmitter configured to transmit an RF or microwave signal into the boiler water and a receiver configured to receive a reflected signal from a reflection of the RF or microwave signal by the boiler water; and
an analysis module configured to determine a conductivity value for the boiler water based on the reflected signal and to determine a TDS concentration for the boiler water based on the conductivity.

9. A method of estimating total dissolved solids (TDS) concentration in boiler water, the method comprising:
transmitting an RF or microwave signal into the boiler water;
receiving a reflected signal from a reflection of the RF or microwave signal by the boiler water;
determining a conductivity value for the boiler water based on the reflected signal; and
determining a TDS concentration for the boiler water based on the conductivity.

10. A method as claimed in claim 9, further comprising:
determining a reflection coefficient based on the reflected signal;
wherein the conductivity value is determined based on the reflection coefficient.

11. A method as claimed in claim 9, wherein the RF or microwave signal is transmitted and received using a sensor probe.

12. A method as claimed in claim 11, wherein the sensor probe is an open-ended coaxial probe.

13. A method as claimed in claim 9, further comprising:
determining if the TDS concentration exceeds a threshold value; and
taking action if the TDS concentration does exceed the threshold value.

14. A method as claimed in claim 13, wherein the action includes providing an alert to an operator.

15. A method as claimed in claim 13, wherein the action includes opening a blowdown control valve in order to reduce the TDS concentration.
